(19) United States
(12) Patent Application Publication
(10) Pub. No.: US 2005/0008613 A1
(43) Pub. Date: Jan. 13, 2005

(54) Antimicrobial quaternary ammonium organosilane coatings

(75) Inventors: William Peterson, Phoenix, AZ (US); Renee E. Berman, Chandler, AZ (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: COATING SYSTEMS LABORATORIES, INC.

(21) Appl. No.: 10/850,121

(22) Filed: May 19, 2004

Related U.S. Application Data
(60) Provisional application No. 60/472,429, filed on May 22, 2003.

(51) Int. Cl. A61K 31/785

(52) U.S. Cl. 424/78.27

(57) ABSTRACT

The present invention provides novel solid phase carriers coated with a quaternary ammonium organosilane coating for use in reducing or eliminating the viable number of microorganisms in a liquid. The invention is useful in a wide variety applications, such as water purification and reduction of harmful microorganisms in liquid foodstuffs.

### CROSS-REFERENCES TO RELATED APPLICATIONS

This application is related to U.S. Provisional Patent Application No. 60/472,429, filed May 22, 2003, and claims all benefits legally available therefrom. Provisional Patent Application No. 60/472,429 is hereby incorporated in its entirety for all purposes capable of being served thereby.

### BACKGROUND OF THE INVENTION

1. Field of the Invention

This invention relates to methods and compositions for reducing the number of microorganisms in a liquid using a solid phase carrier coated with a quaternary ammonium organosilane coating.

2. Description of the Prior Art

Quaternary ammonium organosilanes have been employed in a wide variety of applications. They have been used effectively in eliminating and reducing microorganismal contamination when applied to a variety of surfaces including metal, glass, plastics, rubber, ceramics and fabrics including cellulose, cotton, acetates and nylon.

Commercial quaternary ammonium organosilanes are available as 42% active material in methanol under the trademark Dow Corning 5700 (3-(trimethoxy-silyl)propyldimethyloctadecyl ammonium chloride) by Aegis Environmental Management, Inc. of Midland, Mich. and Requat 1977 (3-(trimethoxysilyl)-propyldidecylmethyl ammonium chloride) by Sanitized Inc. of New Preston, Conn. Octadecyldimethyl(3-trimethoxysilylpropyl) ammonium chloride (Cat. No. SIO6620.0) as a 60% active solution in methanol, tetradecyldimethyl(3-tri-methoxysilylpropyl) ammonium chloride (Cat. No. SIT7090.0) as a 50% solution in methanol and didecylmethyl(3-trimethoxysilylpropyl) ammonium chloride (Cat. No. SID3392.0) as a 42% solution in methanol are offered by Gelest, Inc. of Tullytown, Pa. They are often applied from solvent solutions such as lower alcohols.

Organosilicon ammonium compounds were first taught by Roth in U.S. Pat. No. 3,560,385. The use of these compounds for antimicrobial purposes is taught in U.S. Pat. No 3,730,701 to Isquith et al. They teach that neutral to alkaline pH values were more effective in algae control, but no claims for stability of aqueous solutions were made.

Algae inhibition was also taught in U.S. Pat. Nos. 3,794,736 and 3,860,709. The inhibition of algae growth on solid surfaces such as cellulose acetate following treatment with organosilane quaternary ammonium materials was described by Abbott et al. in U.S. Pat. No. 3,817,739. Abbott et al. in U.S. Pat. No. 3,865,728 again describes algicidal reduction on fibrous materials including polyesters as well as cellulose acetate. G. Kinstedt in U.S. Pat. No. 4,005,025 claims organosilane quaternaries as being useful in imparting soil release properties to hard surfaces in detergent applications such as dishwashing liquids and general hard surface cleaners, but makes no claims for antimicrobial activity.

U.S. Pat. No. 4,005,028 to Heckert et al. similarly teaches detergent compositions containing zwitterionic or ampholytic detergents and quaternized organosilanes. Similarly, U.S. Pat. No. 4,005,030 by D. Heckert and D. Watt teach detergent formulations useful as oven cleaners, window cleaners or toiler bowl cleaners. No antimicrobial claims are present in the '025, '028 and '030 Patents.

In U.S. Pat. No. 4,282,366 organosilicon ammonium compounds are used to render paper substrates resistant to growth of microorganisms. Similarly, Klein in U.S. Pat. No. 4,394,378 claims 3-(trimethoxysilyl)propyldidecylmethyl ammonium chloride as effective against bacteria and fungi on siliceous surfaces, wood, metal, leather, rubber, plastic and textiles.

Other applications for these compounds include paint additives and shoe treatments in U.S. Pat. No. 4,393,378, dentifrice additives in U.S. Pat. No. 4,161,518, and in contact lens disinfectant solution in U.S. Pat. No. 4,615,882. Canadian Patent No. 1,217,004 to Hardy teaches these compounds are antimicrobial when added to bleach such as sodium hypochlorite and can be used as hard surface cleaners.

U.S. Pat. No. 4,406,892 describes treatment of cellulosic fabrics to prevent the growth of disease causing organisms. Similarly, Burril et al. in U.S. Pat. No. 4,421,796 teach a method of treating textile fibers with a mixture of quaternized organosilane compositions in an emulsion containing polydimethylsiloxanes to improve removal of oily soil.

In U.S. Pat. No. 4,467,013 3-(trimethoxysilyl)propyldimethyloctadecyl ammonium chloride is taught as being useful in the treatment of surgical gowns, dressings and bandages. Homan in U.S. Pat. No. 4,564,456 utilizes organosilicon quaternary ammonium compounds to treat water and inhibit corrosion and metal deposition. Hair conditioning compositions containing 3-(trimethoxysilyl)propyl-dimethyloctadecyl ammonium chloride, when applied at pH 8-10 causing the hair to swell is taught by Stadnick in U.S. Pat. No. 4,567,039.

Aqueous emulsions formed by the use of certain organofunctional cationic silanes, including quaternary ammonium organosilanes such as 3-(trimethoxysilyl)propyl-dimethyloctadecyl ammonium chloride, are taught by Blehm et al. in U.S. Pat. No. 4,361,273. The disclosed oil-in-water emulsions allow the transfer of water immiscible liquids and silanes to the surface of certain substrates with the avoidance of any subsequent rewetting or resolubilization of the silane or water immiscible liquid and its subsequent loss from the surface. The water immiscible liquid utilized to prevent resolubilizing of the cationic silane away from the substrate may be silicone oils, waxes, hydrocarbons, glycols or aliphatic alcohols. The preparation of these emulsions utilize co-surfactants such as nonionic and cationic surfactants and require a homogenizer using high shear conditions and teach that the quaternized organosilane does not hydrolyze to the silanol while in its emulsion form and thus does not polymerize to an insoluble siloxane. These emulsions require a water immiscible liquid as described above with which the quaternized organosilane associates, and that a sufficient shear force, requiring specialized mixers, is necessary to form the emulsion they teach. Blehm et al. teaches the prepared emulsion is utilized to transfer the unhydrolyzed quaternized organosilane to the surface where it is protected against resolubilization by the water immiscible component.

The use of similar oil-in-water emulsion compositions in treating acne vulgaris and ringworm is taught in U.S. Pat. No. 4,908,355 to Gettings & White. This patent teaches a method of treating skin disorders through topical application to the epidermis of an oil-in-water emulsion in which there is a quaternary ammonium organosilane, specifically 3-(trimethoxysilyl)propyldimethyloctadecyl ammonium chloride and a low viscosity, low molecular weight water immiscible liquid silicone fluid, which causes the quaternized silane to penetrate the follicular orifices. The volatile silicone fluid is for the purpose of driving the silane into sebaceous glands and destroying the staphylococcal group of bacteria that may reside there. The formation of these emulsions are essentially the formulations taught in the foregoing '273 patent. This patent teaches the quaternized silane can be used neat, in an organic solvent or in aqueous solvent solutions. It further teaches a water immiscible component such as an oil, wax or grease must be present and included in the compositions applied to the skin. It does not teach homogeneous aqueous solutions of the quaternary ammonium organosilane nor demonstrate substantiveness and efficacy on skin. The ability of the silane to be applied directly to the skin from aqueous solutions and the ability for the silane to remain on the skin and continue to be antimicrobially active even after repeated rinsings or washings is not taught.

Glass beads treated with organosilane quaternary compounds are described in U.S. Pat. No. 4,682,992 as being used in air filters where antimicrobial qualities are desired. U.S. Pat. No. 4,781,974 teaches wet towelettes having as organosilicon quaternary ammonium compound substantive to the fibers, but does not teach it being transferred to surfaces or skin. Bryant in U.S. Pat. No. 4,797,420 teaches a formulation containing an alkyl dimethyl benzyl ammonium chloride, a nonionic surfactant and a lower alcohol such as isopropanol in water which is used as a disinfectant formulation.

U.S. Pat. No. 4,847,088 to Blank teaches a quaternary organosilane composition such as 3-(trimethoxysilyl)propyldimethyloctadecyl ammonium chloride when combined with an acid in water will increase the antimicrobial effect. Similar antimicrobial properties are claimed in U.S. Pat. No. 5,013,459 for a method and device to dispense ophthalmic fluids, the porous medium of which has been previously treated with an organosilicon quaternary ammonium material.

U.S. Pat. No. 5,411,585 to Avery et al. teaches further methods for the production of stable hydrolyzable organosilane quaternary ammonium compounds to render surfaces antimicrobial when applied as ingredients in hard surface cleaners.

Stabilized aqueous organosilane solutions, including quaternary ammonium organosilane compounds, with stability of weeks to months, are taught by Elfersy et al. in U.S. Pat. No. 5,954,869. These compositions contain a polyol molecule having at least two hydroxy groups which are separated by no more than three intervening atoms and the organosilane. These compositions may be used to coat a food article, a fluid container or a latex medical article. Sugars are the most common stabilizer taught in this patent.

Antimicrobial skin preparations containing quaternary ammonium organosilanes are taught by Peterson et al. in U.S. Pat. No. 6,613,755, which is herein incorporated by reference in its entirety for all purposes.

The prior art and open literature disclose the antimicrobial properties of quaternary ammonium organosilanes against a wide range of pathogens including, but not limited to: Gram Positive Bacteria such as Citrobacter freundii, Citrobacter diversus, Corynebacterium diptheriae, Diplococcus pneumoniae, Micrococcus sp. (I), Micrococcus sp. (II), Micrococcus sp. (III), Mycobacterium spp., Staphylococcus albus, Staphylococcus aureus, Staphylococcus citrens, Staphylococcus epidermidis, Streptococcus faecalis, and Streptococcus pyogenes; Gram Negative Bacteria such as Acinetobacter calcoaceticus, Enterobacter aerogenes, Enterobacter aglomerans (I), Enterobacter aglomerans (II), Escherichia coli, Klebsiella pneumoniae, Nisseria gonorrhoeae, Proteus mirabilis, Proteus morganii, Proteus vulgaris, Providencia spp., Pseudomonas, Pseudomonas aeruginosa, Pseudomonas fragi, Salmonella choleraesuis, Salmonella enteritidis, Salmonella gallinarum, Salmonella paratyphi A, Salmonella schottmuelleri, Salmonella typhimurium, Salmonella typhosa, Serratia marcescens, Shigella flexnerie Type II, Shigella sonnei, Virbrio cholerae; viruses such Adenovirus Type IV, Feline Pneumonitis, Herpes Simplex Type I & II, HIV-1 (AIDS), Influenza A (Japan), Influenza A2 (Aichi), Influenza A2 (Hong Kong), Parinfluenza (Sendai), Poliovirus, Reovirus, Respiratory Synctia; Fungi and Mold such as, Alternaria alternate, Asperigillus niger, Aureobasidium pullulans, Candida albicans, Cladosporium cladosporioides, Drechslera australiensis, Gliomastix cerealis, Microsporum audouinii, Monilia grisea, Phoma fimeti, Pithomyces chartarum, Scolecobasidium humicola, Trychophyton interdigitale, and Trychophyton mentagrophytes.

The open literature and the prior art cited above do not teach the use of a solid phase carrier coated with a quaternary ammonium organosilane coating to reduce the viable number of microorganisms in a liquid.

### BRIEF SUMMARY OF THE INVENTION

The present invention provides methods and compositions for use in a variety applications, such as water purification and reduction of harmful microorganisms in liquid foodstuffs.

In one aspect, the present invention provides a method of reducing or eliminating the viable number of microorganisms in a liquid. The method includes contacting the liquid with a solid phase carrier coated with a quaternary ammonium organosilane coating.

In an exemplary embodiment, the quaternary ammonium organosilane reagent has the formula:

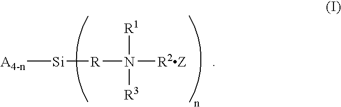

In Formula (I), A is selected from —OR⁴, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Where more than one A is present, each A is independently selected from the groups recited above or below.

R⁴ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R is selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

R¹, R², and R³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

Z is selected from fluoride, chloride, bromide, iodide, tosylate, hydroxide, sulfate and phosphate.

The symbol n is 1, 2 or 3.

### BRIEF DESCRIPTION OF THE DRAWINGS

FIG. 1 illustrates the reduction in the viable number of bacteriophages by quaternary ammonium organosilane coated zeolite.

FIG. 2 illustrates the reduction in the viable number of (A) K. terriena bacteria and (B) E. Coli bacteria by quaternary ammonium organosilane coated zeolite.

FIG. 3 illustrates the average reduction in the viable number of bacteria and bacteriophages by quaternary ammonium organosilane coated zeolite.

FIG. 4 illustrates the reduction in the viable number of algae by quaternary ammonium organosilane coated zeolite.

FIG. 5 illustrates the reduction in the viable number of protozoa parasites by quaternary ammonium organosilane coated zeolite.

FIG. 6 illustrates an experimental apparatus containing a column packed with quaternary ammonium organosilane coated zeolite for use in decreasing the viable number of microorganisms in a liquid.

### DETAILED DESCRIPTION OF THE INVENTION

Definitions

As used herein, the term “reducing the viable number of microorganisms,” means reducing the number of microorganisms capable of growing, working, functioning, and/or developing adequately. The term includes, for example, reducing the overall number of microorganisms, reducing the number of active microorganisms (i.e. inactivating microorganisms), reducing the number of microorganisms able to reproduce, reducing the number of intact microorgansims, reducing the number of infectious agents, removal of microorganisms, inactivation of microorganisms; and/or and the like. “Eliminating the viable number of microorganisms” means reducing the viable number of microorganisms to zero.

The term “microorganism,” as used herein, means an organism that, individually, can only be seen through a microscope. The term microorganism includes, for example, bacteria, fungi, actinomycetes, algae, protozoa, yeast, germs, ground pearls, nematodes, viruses, prions, and algae.

The abbreviations used herein have their conventional meaning within the chemical and biological arts.

Where chemical groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched carbon chain containing at least one carbon, which may be fully saturated, mono- or polyunsaturated. An unsaturated alkyl group is one having one or more double bonds or triple bonds. An “unsubstituted alkyl” refers to branched or unbranched alkyl groups wherein the backbone carbons are attached to hydrogen and/or other backbone carbon. The term “alkylene” refers to a divalent radical derivative of an alkyl.

A “backbone carbon” or “backbone heteroatom,” as used herein, refers to a carbon or heteroatom, respectively, that is not at the point of attachment of an alkyl or heteroalkyl group, and which forms part of a branched or unbranched chain containing at least one carbon.

The term “alkoxy,” refers to those alkyl groups attached to the remainder of the molecule via an oxygen atom.

The term “alkylether” refers to an alkyl having at least one carbon-oxygen-carbon linkage.

The term “hydroxy-substituted alkyl” refers to an alkyl having at least one attached hydroxyl group.

The term “amine-substituted alkyl” refers to an alkyl having at least one attached primary, secondary, or tertiary amine group.

The term “heteroalkyl,” by itself or in combination with another term, means an alkyl having at least one heteroatom within the carbon chain. The heteroatom is selected from the group consisting of O, N, and S, wherein the nitrogen and sulfur atoms may optionally be oxidized and the nitrogen heteroatom may optionally be quaternized. The heteroatom(s) O, N, and S may be placed at any interior position of the heteroalkyl group or at the position at which the alkyl group is attached to the remainder of the molecule. Up to two heteroatoms may be consecutive, such as, for example, —CH₂—NH—OCH₃. Similarly, the term “heteroalkylene” by itself or as part of another substituent means a divalent radical derived from heteroalkyl. For heteroalkylene groups, heteroatoms can also occupy either or both of the chain termini.

An “unsubstituted heteroalkyl” refers to branched or unbranched heteroalkyl groups wherein the backbone carbons are attached to hydrogen, other backbone carbons, and/or backbone heteroatoms. The backbone heteroatoms are attached to hydrogen, backbone carbons, other backbone heteroatoms, and/or oxygen (in the case of oxidized sulfur).

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. The terms “cycloalkylene” and “heterocycloalkylene” refer to the divalent derivatives of cycloalkyl and heterocycloalkyl groups, respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl.

The term “aryl” means, unless otherwise stated, a polyunsaturated, aromatic, hydrocarbon which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the heteroatom occupies a ring vertex (also referred to herein as a “ring heteroatom”). The nitrogen and sulfur atoms are optionally oxidized, and the nitrogen atom(s) are optionally quaternized. A heteroaryl group can be attached to the remainder of the molecule through a carbon or heteroatom. The terms “arylene” and “heteroarylene” refer to the divalent derivatives of aryl and heteroaryl groups, respectively.

An “unsubstituted aryl” or “unsubstituted heteroaryl” refers to aryl and heteroaryl rings, respectively, in which the carbon atoms occupying ring vertices that are not at a point of attachment to the remainder of the molecule are attached only to hydrogen or other atoms occupying ring vertices. Heteroatoms occupying ring vertices that are not at a point of attachment to the remainder of the molecule are attached only to hydrogen, other atoms occupying ring vertices, or oxygen (in the case of oxidized ring heteroatoms).

The term “oxo” as used herein means an oxygen that is double bonded to a carbon atom.

A “liquid,” as used herein, is a substance that flows freely, lacks crystal structure, and, unlike a gas, retains the same volume independent of the shape of its container at ambient temperature and pressure. An “aqueous liquid” refers to a liquid having a portion of water. Aqueous liquids suitable for the practice of the present invention include, for example, waste water and sewage water, fruit juices, milk, and medical fluids. Other suitable fluids will be readily determined by those skilled in the art and are contemplated by the instant invention.

A “solid,” as used herein, is a substance that does not dissolve in water at ambient temperature. Thus, a “solid phase carrier” is a carrier that is insoluble in water at ambient temeperature.

Methods

In one aspect, the present invention provides a method of reducing or eliminating the viable number of microorganisms in a liquid. The method includes contacting the liquid with a solid phase carrier coated with a quaternary ammonium organosilane coating. The quaternary ammonium organosilane coating may reduce the viable number of microorganisms in a liquid by directly contacting the microorganisms.

A wide variety of solid phase carriers are useful in conjunction with the methods and compositions of the present invention. The solid phase carrier may be any appropriate dimension or shape, including, for example, a planar surface, the lining of tubing or pipe, or a roughly spherical particle. The solid phase carrier may also be any appropriate size, including, for example, a microscopic carrier, a carrier detectable with the naked eye, a roughly planar carrier with dimensions that are centimeters to meters in length, and roughly spherical carrier with a radius that is centimeters to meters in length.

The solid phase carrier is typically composed of one or more substance or material that is insoluble in liquid media (e.g. organic media, aqueous media, water, etc.). Exemplary materials include glass, silica, sand (e.g. manganese greensand and filter sand), quartz, flint, zeolite, anthracite, activated carbon, garnet, ilmenite, berm, aluminum (including non-hydrous aluminum silicate (e.g. filter AG)), oxides of iron and titanium (e.g. ilmenite), diatomaceous earth, pozzolan (silicon/alumina material that occurs naturally and is produced as a by product of coal combustion), metal (e.g. tin), ceramic, and/or organic polymers and plastics (e.g. high density polyethylene (HDPE), polypropylene (PP) or polyvinyl chloride (PVC)).

In an exemplary embodiment, the liquid is contacted with an additional solid phase carrier. The additional solid phase character may be coated with a different quaternary ammonium organosilane coating than the solid phase carrier. The additional solid phase carrier may also be composed of a different material than the solid phase carrier.

Quaternary Ammonium Organosilane Reagents

The solid phase carriers of the current invention are coated with a quaternary ammonium organosilane coating. The quaternary ammonium organosilane coating is produced from a quaternary ammonium organosilane reagent. The quaternary ammonium organosilane reagent has the formula:

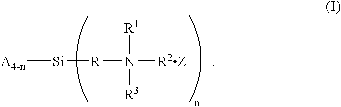

In Formula (I), A is selected from —OR⁴, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. Where more than one A is present, each A is independently selected from the groups recited above or below.

R⁴ is selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

R is selected from substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene.

R¹, R², and R³ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

Z is selected from fluoride, chloride, bromide, iodide, tosylate, hydroxide, sulfate and phosphate.

The symbol n is 1, 2 or 3.

In an exemplary embodiment, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl. For example, where A is a substituted (C₁-C₁₀)alkyl, the substituted (C₁-C₁₀)alkyl is substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl.

In a related embodiment, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl. In another related embodiment, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one substituent independently selected from —OH, unsubstituted (C₁-C₅)alkyl, unsubstituted (C₅-C₇) membered cycloalkyl, and unsubstituted phenyl. In yet another related embodiment, each substituted alkyl, substituted heteroalkyl, substituted cycloalkyl, substituted heterocycloalkyl, substituted aryl, and substituted heteroaryl described herein as possible A, R¹, R², R³, and R⁴ moieties are substituted only with at least one unsubstituted (C₁-C₃)alkyl.

In another exemplary embodiment, each substituted alkylene, substituted heteroalkylene, substituted cycloalkylene, substituted heterocycloalkylene, substituted arylene, and substituted heteroarylene described herein as possible R moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, substituted 5 to 7 membered heterocycloalkyl, unsubstituted aryl, and unsubstituted heteroaryl.

In a related embodiment, each substituted alkylene, substituted heteroalkylene, substituted cycloalkylene, substituted heterocycloalkylene, substituted arylene, and substituted heteroarylene described herein as possible R moieties are substituted only with at least one substituent independently selected from —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl. In another related embodiment, each substituted alkylene, substituted heteroalkylene, substituted cycloalkylene, substituted heterocycloalkylene, substituted arylene, and substituted heteroarylene described herein as possible R moieties are substituted only with at least one substituent independently selected from —OH, unsubstituted (C₁-C₅)alkyl, unsubstituted (C₅-C₇) membered cycloalkyl, and unsubstituted phenyl. In yet another related embodiment, each substituted alkylene, substituted heteroalkylene, substituted cycloalkylene, substituted heterocycloalkylene, substituted arylene, and substituted heteroarylene described herein as possible R moieties are substituted only with at least one unsubstituted (C₁-C₃)alkyl.

A may be selected from —OR⁴, substituted or unsubstituted (C₁-C₁₀)alkyl, substituted or unsubstituted 2 to 12 membered heteroalkyl, substituted or unsubstituted (C₅-C₇)cycloalkyl, substituted or unsubstituted 5 to 7 membered heterocycloalkyl, substituted or unsubstituted aryl, and substituted or unsubstituted heteroaryl. R⁴ may be selected from hydrogen, substituted or unsubstituted (C₁-C₁₀)alkyl, substituted or unsubstituted 2 to 10 membered heteroalkyl, substituted or unsubstituted (C₅-C₇)cycloalkyl, substituted or unsubstituted 5 to 7 membered heterocycloalkyl, substituted or unsubstituted aryl, and substituted or unsubstituted heteroaryl.

In some embodiments, A is selected from —OR⁴, unsubstituted (C₁-C₁₀)alkyl, unsubstituted 2 to 12 membered heteroalkyl, unsubstituted (C₅-C₇)cycloalkyl, unsubstituted 5 to 7 membered heterocycloalkyl, unsubstituted aryl, and unsubstituted heteroaryl. In a related embodiment, A is selected from —OR⁴, unsubstituted (C₁-C₁₀)alkyl, unsubstituted 3 to 12 membered alkylether, unsubstituted (C₅-C₇)cycloalkyl, and unsubstituted phenyl.

A may also be selected from —OR⁴, unsubstituted (C₁-C₄)alkyl, unsubstituted 3 to 8 membered alkylether, unsubstituted (C₅-C₇)cycloalkyl, and unsubstituted phenyl. Alternatively, A is selected from —OR⁴, unsubstituted (C₁-C₄)alkyl, and unsubstituted 3 to 8 membered alkylether.

R⁴ maybe selected from hydrogen, unsubstituted (C₁-C₁₀)alkyl, unsubstituted 2 to 12 membered heteroalkyl, unsubstituted (C₅-C₇)cycloalkyl, unsubstituted 5 to 7 membered heterocycloalkyl, unsubstituted aryl, and unsubstituted heteroaryl.

In some embodiments, R⁴ is selected from hydrogen, unsubstituted (C₁-C₁₀)alkyl, unsubstituted 3 to 12 membered alkylether, unsubstituted (C₅-C₇)cycloalkyl, and unsubstituted phenyl. In a related embodiment, R⁴ is selected from hydrogen, unsubstituted (C₁-C₈)alkyl, unsubstituted 3 to 8 membered alkylether, unsubstituted (C₅-C₇)cycloalkyl, and unsubstituted phenyl. Alternatively, R⁴ is selected from hydrogen, unsubstituted (C₁-C₈)alkyl, and unsubstituted 3 to 8 membered alkylether.

R⁴ may also be selected from phenyl, methylphenyl, substituted or unsubstituted (C₁-C₈)alkyl, and —(CH₂)ₓ—O—(CH₂)_{y}CH₃. X and y are integers independently selected from 1 to 10.

R may be selected from substituted or unsubstituted (C₁-C₁₀) alkylene, substituted or unsubstituted 2 to 10 membered heteroalkyene, substituted or unsubstituted (C₅-C₇)cycloalkyene, substituted or unsubstituted 2 to 7 membered heterocycloalkylene, substituted or unsubstituted arylene, and substituted or unsubstituted heteroarylene.

In an exemplary embodiment, R is a member selected from unsubstituted (C₁-C₁₀)alkylene, unsubstituted 2 to 10 membered heteroalkylene, unsubstituted (C₅-C₇)cycloalkylene, unsubstituted 5 to 7 membered heterocycloalkylene, unsubstituted arylene, and unsubstituted heteroarylene.

R may also be unsubstituted (C₁-C₁₀)alkylene.

R¹, R², and R³ may be selected from hydrogen, substituted or unsubstituted (C₁-C₂₀)alkyl, substituted or unsubstituted 2 to 20 membered heteroalkyl, substituted or unsubstituted (C₅-C₇)cycloalkyl, substituted or unsubstituted 5 to 7 membered heterocycloalkyl, substituted or unsubstituted aryl, and substituted or unsubstituted heteroaryl.

In some embodiments, R¹, R², and R³ are independently selected from hydrogen, unsubstituted (C₁-C₂₀)alkyl, hydroxy-substituted (C₁-C₂₀)alkyl, amine-substituted (C₁-C₂₀)alkyl, unsubstituted 2 to 20 membered heteroalkyl, unsubstituted (C₅-C₇)cycloalkyl, unsubstituted 5 to 7 membered heterocycloalkyl, unsubstituted aryl, and unsubstituted heteroaryl. In a related embodiment, R¹, R², and R³ are independently selected from hydrogen, unsubstituted (C₁-C₂₀)alkyl, unsubstituted alkylether, hydroxy-substituted (C₁-C₂₀)alkyl, amine-substituted (C₁-C₂₀)alkyl, unsubstituted (C₅-C₇)cycloalkyl, and unsubstituted phenyl.

R¹, R², and R³ may also be selected from hydrogen, unsubstituted (C₁-C₂₀)alkyl, unsubstituted alkylether, hydroxy-substituted (C₁-C₂₀)alkyl, amine-substituted (C₁-C₂₀)alkyl, unsubstituted (C₅-C₇)cycloalkyl, and unsubstituted phenyl. Alternatively, R¹, R², and R³ is selected from hydrogen, unsubstituted (C₁-C₂₀)alkyl, unsubstituted alkylether, hydroxy-substituted (C₁-C₂₀)alkyl, and amine-substituted (C₁-C₂₀)alkyl.

In other exemplary embodiments, R¹, R², and R³ are independently selected from —(CH₂)_{q}OCH₃, —(CH₂)_{q}OH, —(CH₂)_{q}O(CH₂)ₜCH₃, —CH₂)_{q}NHCH₃, —(CH₂)_{q}NH₂, —(CH₂)_{q}N(CH₃)₂ and —(CH₂)_{q}NH₂(CH₂)ₜCH₃, in which q and t are integers independently selected from 0 to 10. R¹, R², and R³ may also members independently selected from the group consisting of —CH₂CH₂OCH₃ and —CH₂CH₂OCH₂CH₂CH₃. Alternatively, R¹, R², and R³ may also be independently selected from —CH₂CH₂OH and —CH₂CH₂CH₂CH(OH)CH₃. R¹, R², and R³ may also be independently selected from —CH₂CH₂NH₂ and —CH₂CH₂N(CH₃)₂. Finally, R¹, R², and R³ may be members independently selected from methyl, octadecyl, didecyl, and tetracecyl.

In an exemplary embodiment, the quaternary ammonium organosilane reagent is selected from (CH₃O)₃Si(CH₂)₃N⁺(CH₃)₂(C₁₈H₃₇)(Cl⁻); (CH₃CH₂O)₃Si(CH₂)₃N⁺(CH₃)₂(C₁₈H₃₇)(Cl⁻); (CH₃O)₃Si(CH₂)₃N⁺(CH₃)₂(C₁₈H₃₇)(Br⁻); (CH₃O)₃Si(CH₂)₃N⁺(C₁₀H₂₁)₂(CH₃)(Cl⁻); (CH₃O)₃Si(CH₂)₃N⁺(CH₃)₂(C₁₄H₂₉)(Cl⁻); (CH₃O)₃Si(CH₂)₃N⁺(CH₃)₂(C₁₄H₂₉)(Br⁻); and (CH₃O)₃Si(CH₂)₃N⁺(CH₃)₂(C₁₆H₃₃)(Cl⁻). In a related embodiment, the quaternary ammonium organosilane reagent is selected from 3-(trimethoxysilyl)propyldimethyloctadecyl ammonium chloride, 3-(trimethoxysilyl)propyl-didecylmethyl ammonium chloride, and 3-(trimethoxysilyl)propyldimethyltetradecyl ammonium chloride.

In another exemplary embodiment, the quaternary ammonium organosilane contains an ammonium halide and a hydrolyzable alkoxy group bonded to silicon.

Quaternary Ammonium Organosilane Coatings

A variety of methods may be used to form the quaternary ammonium organosilane coatings from quaternary ammonium organosilane reagents. The quaternary ammonium organosilane reagent may be applied to the solid phase carrier using any method known in the art, including, for example, methods for covalently or non-covalently binding the quaternary ammonium organosilane reagent to the solid phase carrier to form a quaternary ammonium organosilane coating.

Solid phase carriers may be contacted (e.g. sprayed, dipped, or otherwise applied) with a solution preparation containing the quaternary ammonium organosilane reagent. In some embodiments, the quaternary ammonium organosilane reagent coated surfaces are allowed to air dry at room temperatures for a sufficient period of time to complete a condensation cure of the quaternary ammonium organosilane coating. Alternatively, heat is applied to the coated surfaces for a sufficient period of time to effect cure, the duration and temperature of such is known to those skilled in the art.

In an exemplary embodiment, the quaternary ammonium organosilane reagent is covalently bound to the solid phase carrier. Typically, the quaternary ammonium organosilane reagent is covalently bound to an accessible carrier reactive group that forms a part of the solid phase carrier. A variety of reactive groups are useful in covalently binding the quaternary ammonium organosilane reagent. The quaternary ammonium organosilane reagent may be covalently bound to the carrier reactive group through the silane moiety of the quaternary ammonium organosilane reagent. The silane moiety, as used herein, refers to the A₄₋ₙ-Si— portion of the compound of Formula (I).

The silane moiety may be covalently bound to the carrier reactive group by allowing the carrier reactive group to covalently bind to the silicon atom of the silane moiety. For example, where the carrier reactive group is a hydroxyl, the oxygen atom may be allowed to bind to the silicon atom to form a silicon-oxygen bond thereby covalently attaching the quaternary ammonium organosilane reagent to the carrier molecule. In a related embodiment, the silane moiety includes at least one —OR⁴ that leaves upon attack of a hydroxyl carrier reactive group. This reaction may be referred to herein as a condensation reaction. Thus, the quaternary ammonium organosilane reagent may be covalently attached to the carrier molecule via a condensation reaction.

The silane moiety may also include an A group that contains a reactive group, referred to herein as a silane reactive group. The silane reactive group is capable of reacting with a carrier reactive group to form a covalent bond.

Silane reactive groups, carrier reactive groups and classes of reactions useful in covalently attaching quaternary ammonium organosilane reagents to a solid phase carrier are generally those that are well known in the art of bioconjugate chemistry. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful silane and carrier reactive functional groups include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides or reacted with acyl halides;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds; and

(k) phosphoramidites and other standard functional groups useful in nucleic acid synthesis.

The reactive functional groups can be chosen such that they do not participate in, or interfere with, the reactions necessary to assemble the quaternary ammonium organosilane coating. Alternatively, a silane or carrier reactive functional group can be protected from participating in the reaction by the presence of a protecting group. Those of skill in the art will understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

Linkers may also be employed to attach the quaternary ammonium organosilane reagent to the solid phase carrier. Linkers may include reactive groups at the point of attachment to the quaternary ammonium organosilane reagent and/or the solid phase carrier. Any appropriate linker may be used in the present invention, including substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. In an exemplary embodiment, the linker group is selected from substituted or unsubstituted alkylene, and substituted or unsubstituted heteroalkylene. In a related embodiment, the linker is selected from unsubstituted alkylene, alkylene substituted with at least one oxy, unsubstituted heteroalkylene, and heteroalkylene substituted with at least one oxy. In another related embodiment, the linker is selected from unsubstituted (C₁-C₂₅) alkylene, (C₁-C₂₅) alkylene substituted with at least one oxy, unsubstituted 2 to 26 membered heteroalkylene, and 2 to 26 membered heteroalkylene substituted with at least one oxy.

Other useful linkers include those having a polyester backbone (e.g. polyethylene glycol), and derivatives thereof. A wide variety of useful linkers are commercially available (e.g. polyethylene glycol based linkers such as those available from Nektar, Inc. of Huntsville, Ala.).

The quaternary ammonium organosilane reagent may also be non-covalently attached to the solid phase carrier using any interaction, such as Van der Waals interactions, hydrophobic interactions, dipole-dipole interactions, electrostatic interactions, and/or hydrogen bonding interactions.

In an exemplary embodiment, the quaternary ammonium organosilane reagent forms a polymeric network that partially or wholly covers the solid phase carrier. Where the quaternary ammonium organosilane reagent forms a polymeric network, the quaternary ammonium organosilane reagent may additionally from a covalent and/or non-covalent bond with the solid phase carrier.

The quaternary ammonium organosilane reagent typically forms a polymeric network by covalently binding through the silane moiety. Where the silane moiety includes at least one —OR⁴ group, the quaternary ammonium organosilane reagent may form a silicone polymer having a series of silicon-oxygen-silicon bonds. The silicones may be linear polymers or cross-linked polymers. For example, where the silane moiety includes at least two —OR⁴ groups, the quaternary ammonium organosilane reagent may form a cross-linked silicone polymer wherein each silica atom forms part of at least two silicon-oxygen-silicon bonds. Thus, polymerization may be achieved using silane reactive groups capable of forming intermolecular covalent bonds with other silane reactive groups.

In an exemplary embodiment, the quaternary ammonium organosilane reagent is contacted with an aqueous liquid prior to application to the solid phase carrier. As discussed above, useful quaternary ammonium organosilane reagents include those containing hydrolyzable alkoxy groups bound to the silicon atom. Upon contact with a water molecule, the alkoxy groups (e.g. methoxy) may hydrolyze to form hydroxy substituted silicon atoms (also referred to herein as “silanols”) with simultaneous liberation of alcohol as a by-product of the hydrolysis (also referred to herein as condensation). The resultant compound formed on addition of quaternary ammonium organosilanes of the above compositions are the corresponding mono-, di-, or tri-silanol species. The reactive silanol species prepared upon hydrolysis may form covalent silicon-oxygen-silicon bonds with other silanol species resulting in polymeric coatings as described above. The resultant polymeric coating may be a molecular network non-covalently and/or covalently bonded to the solid phase carrier.

It will be understood by those skilled in the art that the quaternary ammonium organosilane coating may form three dimensional, cross-linked, water-insoluble, polymeric coatings which may contain some uncondensed silanol or alkoxy moieties. Monomeric, dimeric and oligomeric species may be present on the solid phase carrier following application of an aqueous solution containing quaternary ammonium organosilane reagent, and these may bond to the solid phase carrier, whether by covalent or non-covalent mechanisms.

The quaternary ammonium organosilane coatings formed on the solid phase carriers retain their antimicrobial activity. They are substantive to the solid phase carriers and largely insoluble in aqueous liquid. For example, in some embodiments, less than 10 ppb of quaternary ammonium organosilane reagents is detectable in water after Standard 42 testing as performed by NSF International, Ann Arbor, Mich.

In an exemplary embodiment, the quaternary ammonium organosilane coating has the formula:

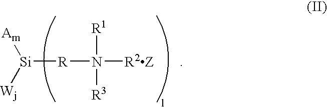

In Formula (II), A, R, R¹, R², and R³ are as defined above in Formula (I). W is a solid phase carrier as described above. The solid phase carrier W may include a linker moiety and/or the remnant of a reactive group. The symbol 1 represents an integer selected from 1, 2, or 3. The symbols m and j represent integers independently selected from 0, 1, 2, and 3, wherein both m and j are not simultaneously 0. The sum of m,j, and 1 is not greater than four. In a related embodiment, 1 is 1, 2, or 3; m is 1, 2, or 3, and j is 1, 2, or 3. In another related embodiment, 1 is 1; m is 1, 2, or 3, and j is 1, 2, or 3.

Microorganisms

The term “microorganism,” as used herein, means an organism that, individually, can only be seen through a microscope. The term microorganism includes, for example, bacteria, fungi, actinomycetes, algae, protozoa, yeast, germs, ground pearls, nematodes, viruses, prions, and algae. Thus, in an exemplary embodiment, the microorganism is selected from bacteria, viruses (also referred to herein as bateriophages), fungi, algae, mold, yeast, spores, and protozoa parasites. The term “bacteria” includes both gram positive and gram negative bacteria.

Gram positive bacteria include, for example, Bacillus sp. (vegetative cell), Corynebacterium diptheriae, Micrococcus lutea, Micrococcus sp., Mycobacterium tuberculosis, Mycobacterium smegmatis, Propionibacterium acnes, Staphylococcus aureus, Staphylococcus epidermidis, Streptococcus faecalis, Streptococcus mutans, Streptococcus pneumonia, and Streptococcus pyogenes.

Gram negative bacteria include, for example, Acinetobacter calcoaceticus, Aeromonas hydrophilia, Citrobacter deversus, Citrobacter freundi, Enterobacter aerogenes, Enterobacter aglomera, Escherichia coli, Klebsiella oxytoca, Klebsiella pneumoniae, Klebsiella terriena, Legionella pneumophila, Morganella morganii, Proteus mirabilis, Proteus vulgaris, Pseudomonas aeruginosa, Pseudomonas fluorscens, Salmonella cholera suis, Salmonella typhi, Salmonella typhimurium, Serratia liquifaciens, and Xanthomonas campestris.

Viruses include, for example, Adenovirus Type II & IV, Bovine Adenovirus Type I & IV, Feline pneumonitis, Herpes Simplex Type I, Herpes Simplex Type II, HIV-1 (AIDS), Influenza A2 (Aichi), Influenza A2 (Asian), Influenza B, Mumps, Parinfluenza (Sendai), Reovirus Type I, Simian Virus 40, Vaccinia, MS2, and PRD1.

Fungi, algae, mold, yeast, and spores include, for example, Alterania alternate, Aspergillus flavus, Aspergillus niger, Aspergillus sydowi, Aspergillus terreus, Aspergillus versicolor, Aspergillus verrucaria, Aureobasidium pullans, Candida albicans, Candida pseudotropocalis, Chaetomium globsum, Cladosporium cladosporioides, Chlorella vulgaris, Dreschslera australiensis, Epidermophyton sp., Gliomastix cerealis, Gloeophyllum trabeum, Microsporum sp., Microsporum audouinii, Monilia grisea, Oscillatoria, Penicillium chrysogenum, Pencillium commune, Penicillium funiculosum, Penicillium pinophiliumm, Penicillium variable, Phoma fimeti, Pithomyces chartarum, Poria placenta, Scenedesmus, Saccharonyces cerevisiae, Scolecobasidium humicola, Trichoderma viride, Trichophyton interdigitale, Trichophyton maidson, Trichophyton mentogrophytes, and Trichophyton sp.

Protozoa parasites include, for example, Cryptosporidium parvum (oocysts) and Giardia.

For more detailed information regarding antimicrobial activity against gram positive bacteria, gram negative bacteria, viruses, fungi, algae, mold, yeast, spores and protozoa parasites, see Hsiao, Y. Chinese Pat. Appl., PCT/CN98/00207 (1998); Malek, J. et al., U.S. Pat. No. 4,259,103 (1981); Klein, S., U.S. Pat. No. 4,394,378 (1983); Eudy, W., U.S. Pat. No. 4,406,892 (1983); Gettings, R. et al., U.S. Pat. No. 4,908,355 (1990) and U.S. Pat. No. 5,013,459 (1991); Blank, L. et al., U.S. Pat. No. 5,145,596 (1992); Avery, R. U.S. Pat. No. 5,411,585 (1995); Blank, L. et al., U.S. Pat. No. 4,865,844 (1989); Battice, D. et al., U.S. Pat. No. 4,631,297 (1986); Higgs, B. et al., U.S. Pat. No. 5,359,104 (1994); Avery, R et al., U.S. Pat. No. 5,411,585 (1995); White, W. et al., Book of Papers, 12^{th} Annual Nonwovens Tech. Symposium, pp. 13-46 (1984); McGee, J. et al., Am. Dyestuff Rep. 6: 56-59 (1983); Dow Coming Technical Brochure; 22-994-83 (1983); Gettings, R. et al., Book of Papers, American Association of Textile Chemists and Colorists National Technical Conference, pp. 259-261 (1978); Dow Corning Technical Brochure, 24-095-85 (1985); Tsao, I. et al., Biotechnol. Bioeng., 34: 639-46 (1989); Tsao, I et al., ACS Synp.Ser. 419: 250-67 (1990); Klein, M. et al., Principles of Viral Inactivation, 3^{rd} Ed., S. Block, Ed., (Lea & Febiger, Philadelphia, Pa.) pp. 422-434 (1983); Peterson, W. et al., U.S. Pat. No. 6,613,755; each of which is incorporated by reference in their entirety for all purposes.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the reagents of the present invention are equally applicable the coatings of the present invention described herein. All publications, patents, and patent applications cited herein are hereby incorporated by reference in their entirety for all purposes.

### EXAMPLES

The following examples are provided by way of illustration only and not by way of limitation. Those of skill in the art will readily recognize a variety of non-critical parameters that can be changed or modified to yield similar results.

ODTA: Octadecyldimethyl(3-trimethoxysilyl)propyl ammonium chloride. Obtained from Wright Chemical Corp., Wilmington, N.C. as a 42% active material in methanol. This material may also be named as 3-(trimethoxysilyl)propyl-dimethyloctadecyl ammonium chloride. Also available as a 42% active material from Aegis Environmental Management, Inc., Midland, Mich. marketed as Dow Coming® 5700.

REQUAT: 3-(trimethoxysilyl)propyldidecylmethyl ammonium chloride. Obtained from Sanitized Inc., New Preston, Conn.; Requat 1977 as a 42% active material in methanol.

TDTA: 3-(trimethoxysilyl)propyltetradecyldimethyl ammonium chloride obtained from Gelest, Inc., Tullytown, Pa., Cat. No. SIT7090.0 as a 50% solution in methanol.

### Example 1

A solution suitable for application was prepared by adding 4 parts ODTA to 100 parts deionized water with stirring. The resulting clear solution was applied to an open, polyvinyl chloride (PVC) flat-type evaporation pan by atomized spray, insuring that all surfaces were thoroughly wetted. The pan is allowed to air dry for 24 hours to cure the quaternary ammonium organosilane reagents to the container surface to form a quaternary ammonium organosilane coating. Water containing bacteria level previously measured at 10⁷ total bacteria/ml using a BIOSPERSE® Test Kit was added to the pan in a ratio of 4.6 grams of water per square inch of surface area. After 30 minutes the water is sampled using a BIOSPERSE® test kit. After incubation, 10⁵ bacteria/ml was measured. Resampling of the test water at 1 hour and 4 hours gave bacterial counts of 10⁴ and <10³, respectively.

### Example 2

A 4 oz. solution prepared according to Example 1 was added to a 1 pint tin-plated metal test container having ¾ inch screw top. The solution was agitated to completely wet the inside surface of the container for 1 minute and then decanted. The test container was allowed to air dry for one hour. Residual vapors were removed by an air purge for 5 minutes and the container was then heated to 105° C. for one hour to cure the quaternary ammonium organosilane reagents to the container surface to form a quaternary ammonium organosilane coating. Water (300 g) having a high bacterial count of 10⁷ bacteria/ml was added to the test container. The test container was allowed to stand one hour at room temperature. After two hours, the test water bacterial level was measured at 10³ bacteria/ml using a BIOSPERSE® test kit.

### Example 3

Two ounce containers of glass, high density polyethylene (HDPE), polypropylene (PP) or polyvinyl chloride (PVC) were treated with an aqueous solution containing 1.5% TDTA. The containers were heated to 100° C. for one hour to cure the quaternary ammonium organosilane reagent to the container surfaces to form a quaternary ammonium organosilane coating. Each container was then rinsed with one oz. of deionized water. One ounce of water containing 10⁵ bacteria/ml was added to each containers and capped. After 24 hours at room temperature, each container was sampled and bacteria measured with a BIOSPERSE® test kit. All containers indicated bacteria counts of 10³ bacteria/ml following incubation for 24 hours.

### Example 4

Coiled aluminum test tubing 8 ft. in length and having an internal diameter of ¼ inch was treated with a solution of 8 parts REQUAT to 100 parts isopropanol. The tube was filled with the solution, sealed and allowed to stand for 15 minutes. The tube was drained and air dried with a stream of compressed air passing through the tube at a rate of 100 ml/minute for 24 hours to cure the quaternary ammonium organosilane reagent to the tubing surfaces to form a quaternary ammonium organosilane coating. An aqueous liquid containing 10⁷ units/ml of bacteria and algae was passed through the coiled aluminum tubing. The aqueous liquid was gravity circulated through the tubing at a rate of 5 ml/minute resulting in contamination of <10³ bacteria/ml.

### Example 5

An antimicrobial solution suitable for treatment of silicaeous surfaces including sand and zeolites was prepared by adding 67.5 grams REQUAT to a stirred solution containing 3.375 kg deionized water and 3 grams of 3-aminopropyltrimethoxysilane. One kg of the clear solution was sprayed onto 50 pounds of #20 white silica pool filter sand over 5 minutes in a rotary mixer. The wetted material was mixed with agitation for an additional hour and allowed to air dry 24 hrs to cure the quaternary ammonium organosilane reagent to the sand surface to form a quaternary ammonium organosilane coating. The treated sand was employed in a recirculating water system to reduce microbial contamination from 10⁷ bacteria/ml to <10³ bacteria/ml in 30 minutes of operation as measured by a BIOSPERSE® test kit.

### Example 6

Zeolites containing approximately 90% clinoptilolite (Ash Meadows Zeolites, LLC) of 20-40 mesh were thoroughly wetted with a solution containing 7 parts ODTA and 93 parts water. The wet zeolites were allowed to air dry 24 hours and then heated 2 hours at 110° C. in a forced air oven to cure the quaternary ammonium organosilane reagent to the zeolite surfaces to form a quaternary ammonium organosilane coating. The treated zeolites were placed in a 2 inch PVC pipe having an overall length of 38 inches. As described below, dechlorinated water containing known quantities of bacteriophages, bacteria, algae and protozoa were passed through the PVC pipe containing the quaternary ammonium organosilane coated zeolites.

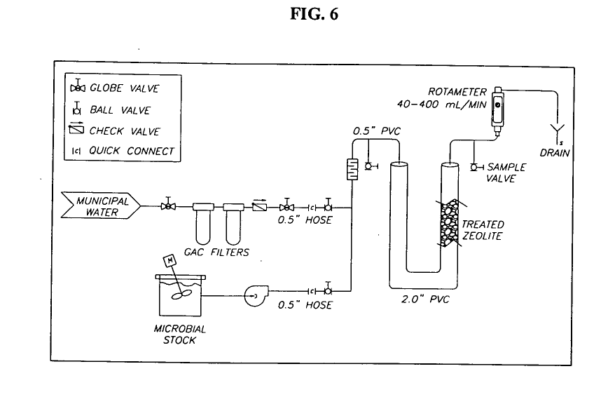

The experimental apparatus consisted of a set of three filters (filter 1, 2 and 3) attached to a manifold, which included fittings for hose connections, and sample ports at the inlet and outlet for each filter (see FIG. 6). An inline mixer was included in the pipe assembly before inlet port to maximize microbial monodispersity. The challenge test water was pumped into each filter at a flow rate of 330 ml/min using a thermally protected pump.

Prior to each microbial challenge, the filters were flushed for 25 minutes with dechlorinated tap water. The flush water was dechlorinated using granular activated carbon filter and chlorine residual was measured before and after the dechlorination using Hach method 8167.

The challenge test water was prepared by adding known number of microorganisms into 20 liters of dechlorinated tap water in a polypropylene container (Nalgene, Rochester, N.Y.). Microbes were washed with 1× phosphate buffered saline just before spiking in the container. The challenge test water container was placed on a stir plate with a Teflon coated stir bar and continuously mixed to provide homogenous distribution of microbes in the influent water. The challenge test water was pumped into each filter using a thermally protected pump (Little Giant Potent Pump, Oklahoma City, Okla.). The pump was primed prior to use by recirculating the microbial stock solution. The hose was connected to the inlet fitting of each filter. The pump was operated for twelve minutes for each filter. The flow rate was measure using a 1000 ml graduated cylinder and adjusted to 330 ml/min as recommended by CSL. Based on the hydraulic parameters of the system, each filter needed a 12-minute-run to stabilize. The effluent samples were taken from each filter after twelve minutes and a single influent sample was collected from the second filter after eight minutes, which represented influent concentration for the complete run. Once the experiment was complete, the filters were again flushed for 30 minutes with dechlorinated tap water.

6.1 Bacteriophages

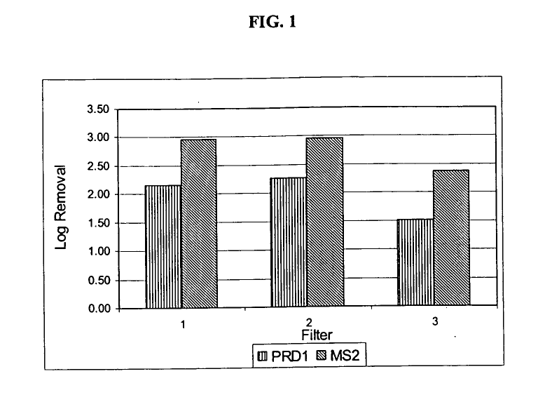

A series of experiments were conducted with the bacteriophages MS2 and PRD1. The effluent and influent samples were taken and diluted as described above. The samples for MS2 and PRD1 were serially diluted and assayed using their respective bacterial hosts by double layer agar method (Adams, M. H., Bacteriophages, Interscience, New York (1959)). The plates were incubated at 37° C. for 24 hours, at which time clear virus plaques were counted. The results are presented in FIG. 1. The log removal and inactivation for MS2 and PRD1 ranged between 2.40 to 2.96, and 1.50 to 2.27 log, respectively. The over average removal for MS2 and PRD1 were 2.8 and 2.0 log, respectively. The data shows that quaternary ammonium organosilane coated zeolite can reduce the viable number of bacteriophages in aqueous liquid.

6.2 Bacteria

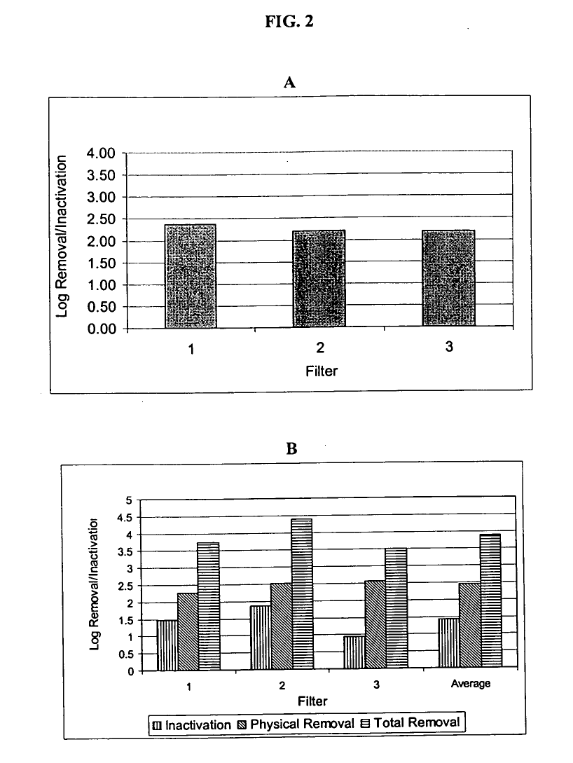

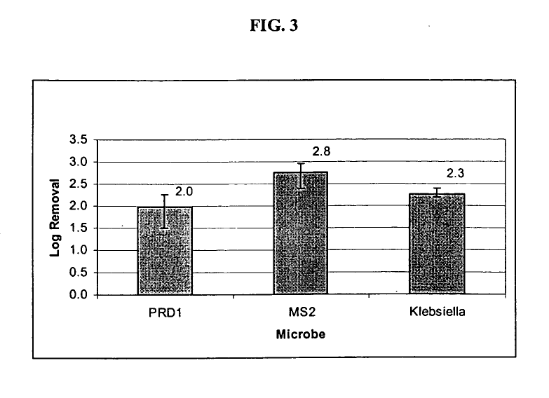

An independent series of experiments were conducted with the bacteria Klebsiella terriena and E. Coli (ATCC 25922). The effluent and influent samples were taken and diluted as described above. The samples were assayed by membrane filtration techniques using 0.4 μm pore size membrane filter. The membrane filter was placed on a selective medium and incubated at 37° C. for 24 hours, at which time bacterial colonies were counted. The results are presented in FIGS. 2(A) and (B). As shown in FIG. 2(A) and FIG. 3, consistent removal for Klebsiella was observed in all the filters, which ranged from 99.37% (2.2 log) to 99.60% (2.4 log) with an average of 99.50% (2.3 log). As shown in FIG. 2(B), the removal for E. coli ranged from 99.96% (3.50 log) to 99.99% (4.39 log) with an average of 99.98% (3.88 log). This study shows that quaternary ammonium organosilane coated zeolite can effectively reduce the viable number bacteria in aqueous liquid.

6.3 Algae

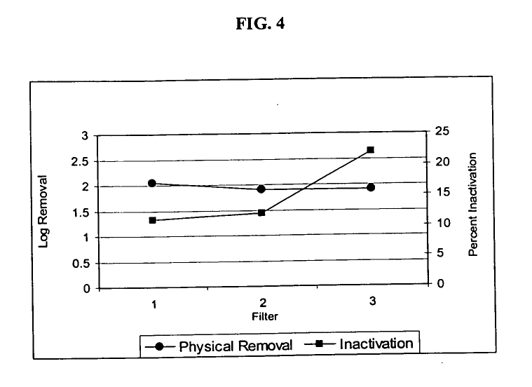

Experiments were conducted with Chorella vulgaris to determine both the removal as well as inactivation effects of the media against algae. The effluent and influent samples were taken and diluted as described above. The samples were concentrated by centrifugation before assaying for total removal and inactivation. Removal was determined by total volumetric counts under microscope. The inactivation rate was determined by viability test. The algal cells were digested with 2% trypsin (in hanks balanced salt solution) and stained with Fluorescein Diacetate (Sigma Chemicals F-7378). Fluorescein Diacetate (FDA) is a non-polar ester that passes through cell membranes. Once inside the cell, FDA is hydrolyzed by esterases (an enzyme present in viable cells) to produce fluorescein, which accumulates inside viable cell walls and fluoresce under UV light. A microscope equipped with both white and ultraviolet light, was used to quantify live and dead algal cells. The results are presented in FIG. 4. The average removal of 99.11% (2.05 log), 98.74% (1.90 log) and 98.74% (1.90 log) were observed for filter 1, 2, and 3, respectively. The average of three inactivation measurements for filter 1, 2, and 3 were 11% (0.05%), 12% (0.06 log) and 22% (0.11 log), respectively. However, based on individual measurements the over all range of inactivation for the three filters was 5% (0.02 log) to 46% (0.27 log) and averaged at 15% (0.07 log). It is clear that quaternary ammonium organosilane coated zeolite can effectively reduce the viable number algae in aqueous liquid.

6.4 Protozoa Parasites

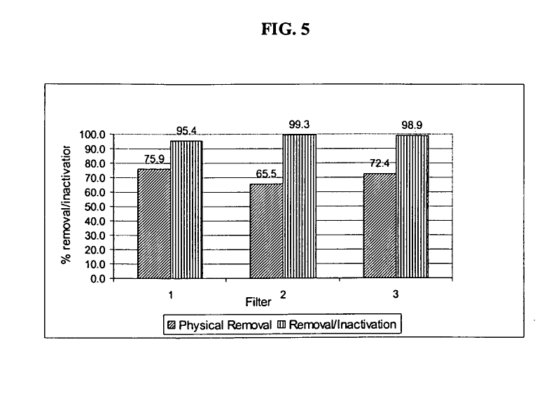

Cryptosporidium parvum oocysts were obtained from the Sterling Parasitology Laboratory at the University of Arizona, Tucson, Ariz., and were used to determine the efficacy of removal or inactivation of infectious oocysts. The removal of Cryptosporidium parvum oocysts was determined by Hemacytometer counts on concentrated samples, whereas, the number of infectious oocysts were determined by infection foci detection method using cell culture technique with the most-probable-number assay (FDM-MPN) (Slifko et al., Applied Environmental Microbiology, 65:3936-3941 (1999). The results are presented in FIG. 5.

The cumulative removal/inactivation of infectious C. parvum oocysts averaged at 97.9% (1.68 log) for all three filters. The removal and inactivation performance by each filter were 95.4% (1.34 log), 99.3% (2.15 log), and 98.9% (1.96 log) for filters 1, 2, and 3, respectively. The removal (only) of oocysts averaged at 71.3% (0.54 log) with an individual removal of 75.9% (0.62 log), 65.5% (0.46 log), and 72.4% (0.56) for filters 1, 2, and 3, respectively. The study indicates that quaternary ammonium organosilane coated zeolite can effectively reduce the viable number protozoa parasites in aqueous liquid.

1. A method of reducing or eliminating the viable number of microorganisms in a liquid, said method comprising contacting said liquid with a solid phase carrier coated with a quaternary ammonium organosilane coating.

2. A method in accordance with claim 1 in which said quaternary organosilane coating is produced from a quaternary ammonium organosilane reagent having the formula: 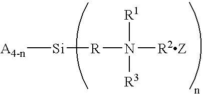
wherein:
A is a member independently selected from the group consisting of —OR⁴, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl,
wherein R⁴ is a member selected from the group consisting of hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
R is a member selected from the group consisting of substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene;
R¹, R², and R³ are members independently selected from the group consisting of hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl;
Z is a member selected from the group consisting of fluoride, chloride, bromide, iodide, tosylate, hydroxide, sulfate and phosphate; and
n is 1, 2 or 3.

3. A method in accordance with claim 1 in which
A is a member independently selected from the group consisting of —OR⁴, substituted or unsubstituted (C₁-C₁₀)alkyl, substituted or unsubstituted 2 to 10 membered heteroalkyl, substituted or unsubstituted (C₅-C₇)cycloalkyl, substituted or unsubstituted 5 to 7 membered heterocycloalkyl, substituted or unsubstituted aryl, and substituted or unsubstituted heteroaryl;
wherein R⁴ is a member selected from the group consisting of hydrogen, substituted or unsubstituted (C₁-C₁₀)alkyl, substituted or unsubstituted 2 to 10 membered heteroalkyl, substituted or unsubstituted (C₅-C₇)cycloalkyl, substituted or unsubstituted 5 to 7 membered heterocycloalkyl, substituted or unsubstituted aryl, and substituted or unsubstituted heteroaryl;
R is a member selected from the group consisting of substituted or unsubstituted (C₁-C₁₀) alkylene, substituted or unsubstituted 2 to 10 membered heteroalkyene, substituted or unsubstituted (C₅-C₇)cycloalkyene, substituted or unsubstituted 5 to 7 membered heterocycloalkylene, substituted or unsubstituted arylene, and substituted or unsubstituted heteroarylene; and
R¹, R², and R³ are members independently selected from the group consisting of hydrogen, substituted or unsubstituted (C₁-C₂₀)alkyl, substituted or unsubstituted 2 to 20 membered heteroalkyl, substituted or unsubstituted (C₅-C₇)cycloalkyl, substituted or unsubstituted 5 to 7 membered heterocycloalkyl, substituted or unsubstituted aryl, and substituted or unsubstituted heteroaryl.

4. A method in accordance with claim 3 in which
each substituted alkyl, substituted heteroalkyl, substituted cycloalkyl, substituted heterocycloalkyl, substituted aryl, and substituted heteroaryl are substituted only with at least one substituent independently selected from the group consisting of —OH, unsubstituted (C₁-C₅)alkyl, unsubstituted 2 to 5 membered heteroalkyl, unsubstituted (C₅-C₇) membered cycloalkyl, unsubstituted 5 to 7 membered heterocycloalkyl, unsubstituted aryl, and unsubstituted heteroaryl; and
said substituted alkylene, substituted heteroalkylene, substituted cycloalkylene, substituted heterocycloalkylene, substituted arylene, and substituted heteroarylene are substituted only with at least one substituent independently selected from the group consisting of —OH, unsubstituted (C₁-C₅)alkyl, unsubstituted 2 to 5 membered heteroalkyl, unsubstituted (C₅-C₇) membered cycloalkyl, substituted 5 to 7 membered heterocycloalkyl, unsubstituted aryl, and unsubstituted heteroaryl.

5. A method in accordance with claim 3 in which
each substituted alkyl, substituted heteroalkyl, substituted cycloalkyl, substituted heterocycloalkyl, substituted aryl, and substituted heteroaryl are substituted only with at least one substituent independently selected from the group consisting of—OH, unsubstituted (C₁-C₅)alkyl, unsubstituted (C₅-C₇) membered cycloalkyl, and unsubstituted phenyl; and
said substituted alkylene, substituted heteroalkylene, substituted cycloalkylene, substituted heterocycloalkylene, substituted arylene, and substituted heteroarylene are substituted only with at least one substituent independently selected from the group consisting of —OH, unsubstituted (C₁-C₅)alkyl, unsubstituted (C₅-C₇) membered cycloalkyl, and unsubstituted phenyl.

6. A method in accordance with claim 3 in which
each substituted alkyl, substituted heteroalkyl, substituted cycloalkyl, substituted heterocycloalkyl, substituted aryl, and substituted heteroaryl are substituted only with at least one unsubstituted (C₁-C₃)alkyl; and
said substituted alkylene, substituted heteroalkylene, substituted cycloalkylene, substituted heterocycloalkylene, substituted arylene, and substituted heteroarylene are substituted only with at least one (C₁-C₃)alkyl.

7. A method in accordance with claim 3 in which
A is a member independently selected from the group consisting of —OR⁴, unsubstituted (C₁-C₁₀)alkyl, unsubstituted 2 to 12 membered heteroalkyl, unsubstituted (C₅-C₇)cycloalkyl, unsubstituted 5 to 7 membered heterocycloalkyl, unsubstituted aryl, and unsubstituted heteroaryl,
wherein R⁴ is a member selected from the group consisting of hydrogen, unsubstituted (C₁-C₁₀)alkyl, unsubstituted 2 to 12 membered heteroalkyl, unsubstituted (C₅-C₇)cycloalkyl, unsubstituted 5 to 7 membered heterocycloalkyl, unsubstituted aryl, and unsubstituted heteroaryl;
R is a member selected from the group consisting of unsubstituted (C₁-C₁₀)alkylene, unsubstituted 2 to 10 membered heteroalkylene, unsubstituted (C₅-C₇)cycloalkylene, unsubstituted 5 to 7 membered heterocycloalkylene, unsubstituted arylene, and unsubstituted heteroarylene;
R¹, R², and R³ are members independently selected from the group consisting of hydrogen, unsubstituted (C₁-C₂₀)alkyl, hydroxy-substituted (C₁-C₂₀)alkyl, amine-substituted (C₁-C₂₀)alkyl, unsubstituted 2 to 20 membered heteroalkyl, unsubstituted (C₅-C₇)cycloalkyl, unsubstituted 5 to 7 membered heterocycloalkyl, unsubstituted aryl, and unsubstituted heteroaryl.

8. A method in accordance with claim 3
A is a member independently selected from the group consisting of —OR⁴, unsubstituted (C₁-C₁₀)alkyl, unsubstituted 3 to 12 membered alkylether, unsubstituted (C₅-C₇)cycloalkyl, and unsubstituted phenyl,
wherein R⁴ is a member selected from the group consisting of hydrogen, unsubstituted (C₁-C₁₀)alkyl, unsubstituted 3 to 12 membered alkylether, unsubstituted (C₅-C₇)cycloalkyl, and unsubstituted phenyl;
R is unsubstituted (C₁-C₁₀)alkylene;
R¹, R², and R³ are members independently selected from the group consisting of hydrogen, unsubstituted (C₁-C₂₀)alkyl, unsubstituted alkylether, hydroxy-substituted (C₁-C₂₀)alkyl, amine-substituted (C₁-C₂₀)alkyl, unsubstituted (C₅-C₇)cycloalkyl, and unsubstituted phenyl.

9. A method in accordance with claim 3 in which
A is a member independently selected from the group consisting of —OR⁴, unsubstituted (C₁-C₄)alkyl, unsubstituted 3 to 8 membered alkylether, unsubstituted (C₅-C₇)cycloalkyl, and unsubstituted phenyl,
wherein R⁴ is a member selected from the group consisting of hydrogen, unsubstituted (C₁-C₈)alkyl, unsubstituted 3 to 8 membered alkylether, unsubstituted (C₅-C₇)cycloalkyl, and unsubstituted phenyl;
R is unsubstituted (C₁-C₁₀)alkylene;
R¹, R², and R³ are members independently selected from the group consisting of hydrogen, unsubstituted (C₁-C₂₀)alkyl, unsubstituted alkylether, hydroxy-substituted (C₁-C₂₀)alkyl, amine-substituted (C₁-C₂₀)alkyl, unsubstituted (C₅-C₇)cycloalkyl, and unsubstituted phenyl.

10. A method in accordance with claim 3 in which
A is a member independently selected from the group consisting of —OR⁴, unsubstituted (C₁-C₄)alkyl, and unsubstituted 3 to 8 membered alkylether,
wherein R⁴ is a member selected from the group consisting of hydrogen, unsubstituted (C₁-C_{8 )alkyl, and unsubstituted} 3 to 8 membered alkylether;
R is unsubstituted (C₁-C₁₀)alkylene;
R¹, R², and R³ are members independently selected from the group consisting of hydrogen, unsubstituted (C₁-C₂₀)alkyl, unsubstituted alkylether, hydroxy-substituted (C₁-C₂₀)alkyl, and amine-substituted (C₁-C₂₀)alkyl.

11. A method in accordance with claim 10 in which R¹, R², and R³ are members independently selected from the group consisting of —(CH₂)_{q}OCH₃, —(CH₂)_{q}OH, —(CH₂)_{q}O(CH₂)ₜCH₃, —CH₂)_{q}NHCH₃, —(CH₂)_{q}NH₂, —(CH₂)_{q}N(CH₃)₂ and —(CH₂)_{q}NH₂(CH₂)ₜCH₃, wherein q and t are integers independently selected from 0 to 10.

12. A method in accordance with claim 10 in which R¹, R², and R³ are members independently selected from the group consisting of —CH₂CH₂OCH₃ and —CH₂CH₂OCH₂CH₂CH₃.

13. A method in accordance with claim 10 in which R¹, R², and R³ are members independently selected from the group consisting of —CH₂CH₂OH and —CH₂CH₂CH₂CH(OH)CH₃.

14. A method in accordance with claim 10 in which R¹, R², and R³ are members independently selected from the group consisting of —CH₂CH₂NH₂ and —CH₂CH₂N(CH₃)₂.

15. A method in accordance with claim 10 in which R¹, R², and R³ are members independently selected from the group consisting of methyl, octadecyl, didecyl, and tetradecyl.

16. A method in accordance with claim 2 in which said quaternary ammonium organosilane reagent is 3-(trimethoxysilyl)propyldimethyloctadecyl ammonium chloride.

17. A method in accordance with claim 2 in which said quaternary ammonium organosilane reagent is 3-(trimethoxysilyl)propyldidecylmethyl ammonium chloride.

18. A method in accordance with claim 2 in which said quaternary ammonium organosilane reagent is 3-(trimethoxysilyl)propyldimethyltetradecyl ammonium chloride.

19. A method in accordance with claim 1 in which said liquid is contacted with at least one additional solid phase carrier coated with a different quaternary ammonium organosilane coating.

20. A method in accordance with claim 1 in which said microorganisms are member selected from the group consisting of gram-positive and gram-negative bacteria.

21. A method in accordance with claim 1 in which said microorganisms are viruses.

22. A method in accordance with claim 1 in which said microorganisms are fungi.

23. A method in accordance with claim 1 in which said microorganisms are a member selected from the group consisting of algae and mold.

24. A method in accordance with claim 1 in which said microorganisms are yeast.

25. A method in accordance with claim 1 in which said microorganisms are spores.

26. A method in accordance with claim 1 in which said liquid is a member selected from the group consisting of water and aqueous solution.

27. A method in accordance with claim 26 in which said solid phase carrier comprises a material selected from the group consisting of glass, silica, sand, quartz, flint, zeolite, anthracite, activated carbon, garnet, ilmenite, berm, non-hydrous aluminum silicate, oxides of iron and titanium, diatomaceous earth, pozzolan, metal, ceramic, and organic plastic.

28. A method in accordance with claim 1 in which said liquid is contacted with an additional solid phase carrier, wherein said additional solid phase carrier comprises a different material than said solid phase carrier.
